(19) United States
(12) Patent Application Publication
(10) Pub. No.: US 2022/0380936 A1
(43) Pub. Date: Dec. 1, 2022

(54) ENCAPSULATION OF EUKARYOTIC CELLS FOR CELLULAR SCREENING OF EXPRESSED SEQUENCES

(71) Applicant: The Florey Institute of Neuroscience and Mental Health, Parkville (AU)

(72) Inventors: Daniel James Scott, Brunswick West (AU); Ross Bathgate, Coburg (AU)

(21) Appl. No.: 17/818,897

(22) Filed: Aug. 10, 2022

Related U.S. Application Data
(63) Continuation of application No. 16/612,132, filed on Nov. 8, 2019, now abandoned.

(30) Foreign Application Priority Data
May 11, 2017 (AU) 2017901747

(51) Int. Cl. C40B 30/04; C40B 20/00; C40B 40/02; G01N 33/68

(52) U.S. Cl. CPC C40B 30/04; C40B 20/00; C40B 40/02; G01N 33/6803

(57) ABSTRACT

Methods for selecting polypeptides or proteins having one or more desired properties from a library of sequences expressed in eukaryotic cells, including: encapsulating the cells by photopolymerization; solubilizing the encapsulated cells to produce semipermeable microcapsules; optionally contacting the cells and/or the microcapsules with one or more agents to facilitate detection of activity or function of polypeptides or proteins of interest; and selecting polypeptides or proteins of interest having one or more desired properties. Also provided are methods for encapsulating eukaryotic cells for use in the selection of polypeptides and proteins as described above.

### CROSS REFERENCE TO RELATED APPLICATIONS

This application is a continuation of U.S. patent application Ser. No. 16/612,132, filed Nov. 8, 2019, which is a U.S. § 371 National Phase of International Patent Application No. PCT/AU2018/050442, International Filing Date May 11, 2018, which claims priority to Australian Patent Application No. 2017901747, filed on May 11, 2017, the contents of which are incorporated by reference in their entirety.

### FIELD OF THE DISCLOSURE

The present disclosure relates generally to an improved method for encapsulating eukaryotic cells for use in cell-based nucleic acid and protein screening methods.

### BACKGROUND

Directed evolution is a powerful tool for generating nucleic acids and molecules they encode with specific properties. Directed evolution is used for the generation of enzymes and other proteins with improved, altered or novel characteristics and/or functions for a variety of industrial, therapeutic and research applications. For example, proteins may be selected for improved or altered solubility, pH stability, thermostability, detergent stability, folding properties, binding characteristics, improved performance and/or novel functionalities.

A number of techniques and approaches have been developed to facilitate the screening of large libraries of sequences and selecting expressed sequences for desired phenotypes, including phage display, bacterial display, yeast display, ribosome display and in vitro compartmentalization. One technique of particular application in the directed evolution of polypeptides and proteins is a cell-based system known as Cellular High-throughput Encapsulation Solubilization and Screening (CHESS) (see Scott and Plückthun, Direct molecular evolution of detergent-stable G protein-coupled receptors using polymer encapsulated cells. J Mol Biol, 2013, 425:662-677; Yong and Scott, Rapid directed evolution of stabilized proteins with cellular high-throughput encapsulation solubilization and screening (CHESS). Biotechnol Bioeng, 2015, 112:438-446) in which bacterial cells are encapsulated using alternate layers of oppositely charged polymers such as chitosan and alginate. Following encapsulation, the cells are solubilized, leaving the polymeric capsule as a semipermeable barrier allowing the entry of small molecules (such as ligands and detectable labels) and preventing the leakage of larger polypeptides and proteins.

A method for utilising CHESS in the selection of sequences from a library of expressed nucleic acid sequences is described in patent application WO 2013/104686, the disclosure of which is incorporated herein by reference in its entirety. The method disclosed therein is of particular application in bacterial cells. However bacterial cells are typically incapable of performing the same post-translational modifications of proteins performed in eukaryotic cells, and are thus of limited use in the screening and selection of expressed eukaryotic, and in particular human, proteins. Moreover, the present inventors have discovered that the method of cellular encapsulation described and taught in WO 2013/104686, using alternate layers of a cationic polysaccharide (such as chitosan) and an anionic polysaccharide (such as alginate), fails to encapsulate eukaryotic cells.

There remains a need for a method for encapsulating eukaryotic cells to facilitate and expand cellular screening and selection methods such as that described in WO 2013/104686.

### SUMMARY OF THE DISCLOSURE

According to a first aspect of the present disclosure there is provided a method for selecting polypeptides or proteins having one or more desired properties from a library of sequences expressed in eukaryotic cells, comprising:
(i) encapsulating said cells by photopolymerization;
(ii) solubilizing said encapsulated cells to produce semipermeable microcapsules;
(iii) optionally contacting said cells and/or said microcapsules with one or more agents to facilitate detection of activity or function of polypeptides or proteins of interest; and
(iv) selecting polypeptides or proteins of interest having one or more desired properties.

In one embodiment step (iii) occurs prior to step (i) or step (ii), concurrently with step (i) or step (ii), or subsequent to step (ii).

In one embodiment said contacting comprises two or more contacting steps, with the same or different agents, and wherein at least one contacting step occurs prior to said encapsulating or solubilizing and at least one contacting step occurs subsequent to said encapsulating or solubilizing.

In one exemplary embodiment, said contacting may comprise contacting said microcapsules with a ligand or substrate that binds to the polypeptide or protein of interest.

In exemplary embodiments the eukaryotic cells are insect cells or mammalian cells. The mammalian cells may be human cells. In one exemplary embodiment, the human cells are human embryonic kidney cells.

The photopolymerization may comprise photopolymerization of a poly(ethylene glycol) (PEG)-based monomer such as a PEG-diacrylate. The photoinitiator may comprise a dye such as an eosin dye. The eosin dye may be eosin Y. The photopolymerization may be carried out in the presence of an amine, such as triethanolamine, and/or an accelerator such as 1-vinyl-2-pyrrolidinone. In an exemplary embodiment the photoinitiator system comprises an eosin dye, triethanolamine and 1-vinyl-2-pyrrolidinone.

In an exemplary embodiment the encapsulation step comprises:
treating the cells with the photoinitiator, optionally eosin Y;
washing the cells;
treating the cells with a solution comprising a photopolymerizable residue, optionally a PEG-diacrylate, in the presence of an amine, optionally triethanolamine; and
exposing the cells to a visible light source, optionally in the presence of an accelerator such as 1-vinyl-2-pyrrolidinone, to encapsulate the cells.

The method may further comprise the step of subjecting the microcapsules to one or more environmental conditions prior to the selecting step. The environmental conditions may comprise, for example, detergent treatment, temperature, chemical denaturant, or pH. The desired properties typically comprise stability under one or more environmental conditions. The stability may comprise detergent stability, thermostability, chemical stability, or pH stability.

The solubilization step may comprise treating the encapsulated cells with one or more detergents.

According to a second aspect of the present disclosure there is provided a method for encapsulating one or more eukaryotic cells for use in a method for selecting polypeptides or proteins having one or more desired properties from a library of sequences expressed in said eukaryotic cells, wherein said encapsulating comprises photopolymerization.

In exemplary embodiments the eukaryotic cells are insect cells or mammalian cells. The mammalian cells may be human cells. In one exemplary embodiment, the human cells are human embryonic kidney cells.

The photopolymerization may comprise photopolymerization of a poly(ethylene glycol) (PEG)-based monomer such as a PEG-diacrylate. The photoinitiator may comprise a dye such as an eosin dye. The eosin dye may be eosin Y. The photopolymerization may be carried out in the presence of an amine, such as triethanolamine, and/or an accelerator such as 1-vinyl-2-pyrrolidinone. In an exemplary embodiment the photoinitiator system comprises an eosin dye, triethanolamine and 1-vinyl-2-pyrrolidinone.

In an exemplary embodiment the encapsulation step comprises:
treating the cells with the photoinitiator, optionally eosin Y;
washing the cells;
treating the cells with a solution comprising a photopolymerizable residue, optionally a PEG-diacrylate, in the presence of an amine, optionally triethanolamine; and
exposing the cells to a visible light source, optionally in the presence of an accelerator such as 1-vinyl-2-pyrrolidinone,
   to thereby encapsulate the cells.

### BRIEF DESCRIPTION OF THE DRAWINGS

Embodiments of the present disclosure are described herein, by way of non-limiting example only, with reference to the following drawings:

FIG. 1. Light microscopy of human embryonic kidney (HEK) cells. A. Naked (unencapsulated) HEK cells. B. Naked HEK cells of A, diluted approximately 100 fold and treated with 3-[(3-cholamidopropyl)dimethylammonio]-1-propanesulfonate (CHAPS) for 24 hours. C. HEK cells encapsulated according to an embodiment of the present disclosure. D. Encapsulated HEK cells of C, diluted approximately 100 fold and treated with CHAPS for 24 hours.

FIG. 2. Flow cytometry analysis of GFP-expressing HEK cells, naked (unencapsulated) or encapsulated according to an embodiment of the present disclosure (encaped). A, Cell concentration. B, Solubilization (loss of GFP). Numbers in parentheses indicate cell concentration of samples.

FIG. 3. Flow cytometry analysis of neurotensin-binding to NTS1-expressing HEK cells encapsulated according to an embodiment of the present disclosure. A, absence of detergent. B, treated with n-decyl-β-D-maltopyranoside (DM) for 24 hours at 25° C.

FIG. 4. Flow cytometry analysis of naked (unencapsulated) HEK cells, naked cells which have been incubated with dragon-green labelled nanobeads, cells encapsulated according to an embodiment of the present disclosure, and cells co-encapsulated with dragon-green labelled nanobeads according to an embodiment of the present disclosure.

FIG. 5. Fluorescence microscope image of dried dragon green nanobeads, mostly aggregated.

FIG. 6. Transmitted light and fluorescence microscope images of methanol-fixed HEK cells. A and B. Naked cells. C and D. Naked cells which have been incubated with dragon green labelled nanobeads but not encapsulated. E and F. Cells encapsulated according to an embodiment of the present disclosure. G and H. Cells co-encapsulated with dragon green nanobeads according to an embodiment of the present disclosure.

FIG. 7. Flow cytometry analysis of HEK cells encapsulated according to an embodiment of the present disclosure, co-encapsulated with dragon green labelled nanobeads according to an embodiment of the present disclosure, and co-encapsulated followed by treatment with detergent n-dodecyl-β-D-maltopyranoside (DDM) for 3 hours.

FIG. 8. A. Transmitted light microscopy image and B. Fluorescence microscopy image of HEK cells co-encapsulated with dragon green labelled nanobeads according to an embodiment of the present disclosure and treated with detergent (DDM) for 24 hours.

### DETAILED DESCRIPTION

Throughout this specification, unless the context requires otherwise, the word “comprise”, or variations such as “comprises” or“comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the term “about” is understood to refer to a range of numbers that a person of skill in the art would consider equivalent to the recited value in the context of achieving the same function or result.

In the context of this specification, the terms “a” and “an” refer to one or to more than one (i.e. to at least one) of the grammatical object of the article. By way of example, “an element” means one element or more than one element.

The term “polypeptide” means a polymer made up of amino acids linked together by peptide bonds. The term “protein” may also be used to refer to such a polymer although in some instances a polypeptide may be shorter (i.e. composed of fewer amino acid residues) than a protein. Nevertheless, the terms “polypeptide” and “protein” may be used interchangeably herein.

The present disclosure overcomes a disadvantage identified by the inventors with the selection method described and taught in WO 2013/104686 and the limitation of this method to the encapsulation of bacterial cells. For the expression of eukaryotic polypeptides and proteins, and the selection of eukaryotic polypeptides and proteins having desired properties, it is preferable to express the polypeptides and proteins in eukaryotic cells. Thus, suitable means of encapsulating eukaryotic cells are required to enable the selection of polypeptides and proteins having desired properties according to the method described and taught in WO 2013/104686.

Accordingly, provided herein is a method for selecting polypeptides or proteins having one or more desired properties from a library of sequences expressed in eukaryotic cells, comprising:
(i) encapsulating said cells by photopolymerization;
(ii) solubilizing said encapsulated cells to produce semipermeable microcapsules;
(iii) optionally contacting said cells and/or said microcapsules with one or more agents to facilitate detection of activity or function of polypeptides or proteins of interest; and
(iv) selecting polypeptides or proteins of interest having one or more desired properties.

Also provided herein is a method for encapsulating one or more eukaryotic cells for use in a method for selecting polypeptides or proteins having one or more desired properties from a library of sequences expressed in said eukaryotic cells, wherein said encapsulating comprises photopolymerization.

The methods of the present disclosure may be practiced using any eukaryotic cell. Suitable eukaryotic cells include, but are not limited to, yeast cells, protozoal cells, algal or other plant cells, or an animal cells, such as insect or mammalian cells. The cell may be a primary or secondary cell culture or an immortalized cell line. In exemplary embodiments the eukaryotic cell is an insect cell or cell line or a mammalian cell or cell line, optionally a human cell or cell line. The human cell or cell line may be, for example, an embryonic or stem cell or cell line. However those skilled in the art will appreciate that any eukaryotic cell may be employed, and scope of the present disclosure is not limited by the identity or origin of the eukaryotic cell selected for any particular application.

In accordance with the present disclosure encapsulation of the eukaryotic cell is by means of photopolymerization. Those skilled in the art will be familiar with the principles of photopolymerization (see, for example, Baroli, Photopolymerization of biomaterials: issues and potentialities in drug delivery, tissue engineering and cell encapsulation technologies, J Chem Technol Biotechnol, 2006, 81:491-499), and the application of photopolymerization in the context of the present disclosure is well within the capabilities of the skilled person with no undue burden of experimentation. In a broad sense photopolymerization requires a polymerizable monomer (photopolymerizable residue), a photoinitiator and a source of light. In the context of the present disclosure, any suitable photopolymerizable residue, photoinitiator and source of light may be employed, depending on the particular application. The light source may comprise, for example, UV light, visible light or infrared light, depending on the photoinitiator(s) and photopolymerizavble residue(s) used.

By way of example, the photopolymerizable residue may be selected from (di)methacrylic or (di)acrylic derivatives of poly(ethylene glycol) (PEG) and its derivatives, poly (ethylene oxide), poly(vinyl alcohol) (PVA) and its derivatives, PEG-polystyrene copolymers (PEG)-(PST), ethylene glycol-lactic acid copolymers (nEGmLA; where n and in are the number of repeat units of EG and LA, respectively), ethylene glycol-lactic acid-caprolactone copolymers (nEGmLAz CL), PLA-b-PEG-b-PLA, PLA-g-PVA, poly(D,L-lactide-co-ε-caprolactone), (poly)-anhydrides, urethanes, dextran, collagen, and diethyl fumarate/poly(propylene fumarate).

In an exemplary embodiment, the photopolymerizable residue is a PEG-diacrylate. The molecular weight of the PEG may be, for example, between about 1000 Da and about 30,000 Da, of between about 2000 Da and about 8000 Da. For example the molecular weight of the PEG may be about 1000, 1500, 2000, 2500, 3000, 3500, 4000, 4500, 5000, 5500, 6000, 6500, 7000, 7500, 8000, 8500, 9000, 9500, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, or 30,000 Da. In an exemplary embodiment the PEG-diacrylate is PEG-diacrylate 6K, with a molecular weight of approximately 6000 Da. The molecular weight of the PEG may affect the encapsulation efficiency and/or the polymer thickness. The skilled addressee can determine, by routine experimentation only, the optimal molecular weight depending on a variety of factors including the cells used and the particular application of the method.

By way of example, the photoinitiator may be selected from eosin (such as eosin Y), 1-cyclohexyl phenyl ketone, 2,2-dimethoxy-2-phenylacetophenone (DMPA), 2-hydroxy-1-[4-(hydroxyethoxy) phenyl]-2-methyl-1-propanone, or camphorquinone/amine, where the amine is, for example, triethylamine, triethanolamine, or ethyl 4-N,N-dimethylaminobenzoate.

In an exemplary embodiment, the photoinitiator comprises a dye such as eosin Y and/or triethanolamine. The photoinitiator system may comprise eosin Y and triethanolamine. The eosin Y may be used at a concentration of, for example, between about 5 mM and about 500 μM. For example, the eosin Y concentration may be about 5 mM, 20 mM, 50 mM, 100 mM, 250 mM, 500 mM, 750 mM, 1 μM, 50 μM, 100 μM, 150 μM, 200 μM, 250 μM, 300 μM, 350 μM, 400 μM, 450 μM or 500 μM. In an exemplary embodiment the eosin Y is used at a concentration of about 100 μM. The concentration of the eosin Y may affect the encapsulation efficiency. The skilled addressee can determine, by routine experimentation only, the optimal concentration depending on a variety of factors including the cells used and the particular application of the method.

The triethanolamine may be used at a concentration of, for example, between about 100 mM and about 500 mM. For example, the triethanolamine concentration may be about 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, or 500 mM. In an exemplary embodiment the triethanolamine is used at a concentration of about 225 mM. The concentration of the triethanolamine may affect the encapsulation efficiency and/or the polymer thickness. The skilled addressee can determine, by routine experimentation only, the optimal concentration depending on a variety of factors including the cells used and the particular application of the method.

The photopolymerization may be carried out in the presence of an accelerator, such as 1-vinyl-2-pyrrolidinone. The 1-vinyl-2-pyrrolidinone may be used at a concentration of, for example, between about 15 mM and about 100 mM. For example, the 1-vinyl-2-pyrrolidinone concentration may be about 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In an exemplary embodiment the 1-vinyl-2-pyrrolidinone is used at a concentration of about 37 mM. The concentration of the 1-vinyl-2-pyrrolidinone may affect the encapsulation efficiency and/or the polymer thickness. The skilled addressee can determine, by routine experimentation only, the optimal concentration depending on a variety of factors including the cells used and the particular application of the method.

In an exemplary embodiment, encapsulated cells are formed by photopolymerizing a PEG-diacrylate prepolymer solution by initiation with eosin Y and triethanolamine upon illumination with visible light using 1-vinyl-2-pyrrolidinone as an accelerator. In such an embodiment, the photoinitiator system, for photopolymerization of the PEG-diacrylate, may be considered to comprise the eosin dye, the triethanolamine and the 1-vinyl-2-pyrrolidinone. In a particular exemplary embodiment, the encapsulation comprises:
treating the cells with the photoinitiator, optionally eosin Y;
washing the cells;
treating the cells with a solution comprising a photopolymerizable residue, optionally a PEG-diacrylate, in the presence of an amine, optionally triethanolamine; and
exposing the cells to a visible light source, optionally in the presence of an accelerator such as 1-vinyl-2-pyrrolidinone, to encapsulate the cells.

In an exemplary embodiment, the process of encapsulating cells by photopolymerization comprises co-encapsulation with an encapsulation indicator, such that the resulting encapsulating layer comprises both the photopolymerized polymer and the encapsulation indicator. Incorporation of an encapsulation indicator into the encapsulating layer allows successful encapsulation in the resulting cells to be verified by observation of the indicator, and for encapsulated cells to be detected in mixed samples of encapsulated and unencapsulated cells. By way of example only, the encapsulation indicator may comprise labelled nanobeads, for example fluorescently-labelled polystyrene nanobeads. When a cell is co-encapsulated with the nanobeads, the nanobeads are incorporated into the encapsulating layer and remain associated with the cell even after washing. Observance of the fluorescence of the nanobead can then be used to verify that cells have been successfully encapsulated. In an exemplary embodiment, co-encapsulation is carried out by addition of the encapsulation indicator to the photopolymerizable residue before photopolymerization takes place.

In a particular exemplary embodiment, the method for selecting polypeptides or proteins having one or more desired properties from a library of sequences expressed in eukaryotic cells comprises:
encapsulating said cells by photopolymerization;
solubilizing said encapsulated cells to produce semipermeable microcapsules;
optionally contacting said cells and/or said microcapsules with one or more agents to facilitate detection of activity or function of polypeptides or proteins of interest; and
selecting polypeptides or proteins of interest having one or more desired properties,
   wherein the step of encapsulating the cells by photopolymerization comprises co-encapsulating the cells with an encapsulation indicator.

The methods for selecting polypeptides or proteins having one or more desired properties from a library of sequences expressed in eukaryotic cells, in which the encapsulation methods described above may be applied, may be any suitable cellular high-throughput encapsulation solubilization screening (CHESS) method, such as that described and taught in WO 2013/104686, the disclosure of which is incorporated herein in its entirety by reference. Thus, methods and approaches to library construction, solubilization of encapsulated cells, and selection of the polypeptides or proteins of interest having desired properties (such as by detection of ligand or other substrate binding to the polypeptides or proteins of interest) that are described in WO 2013/104686 and equally applicable to the present disclosure.

Broadly speaking, CHESS as originally conceived involves 1) transforming a gene library encoding variant proteins into cells and expressing the proteins in the cells; 2) encapsulating the cells; 3) solubilizing or permeabilizing the cell membrane with detergent; 4) contacting the protein(s) with a ligand (e.g. labelled ligand or enzyme substrate), wherein the encapsulation layer now serves as a semipermeable barrier that retains the protein variant and its encoding gene within the capsule but allows the ligand into the capsule, where it can bind to functional protein; 5) sorting the capsules, for example by FACs, wherein capsules containing variants that bind strongly to the ligand (i.e. retain activity) display stronger detectable signals (e.g. contain more labelled ligands); 6) recovering the genes from the sorted capsules; and 7) identifying the encoded/desired variant protein and/or using the gene as a template for further rounds of mutation or selection. CHESS was originally designed as a high-throughput method to identify detergent-stable G protein-coupled receptors (GPCRs). However, it is a method that can be applied to the directed evolution of any protein, soluble or membrane-bound, including integral membrane proteins, ion channels, enzymes, nuclear receptors, transcription factors, DNA/RNA-binding proteins, antibodies and fragments thereof (e.g. a diabody, a Fab, a Fab′, a F(ab′)₂, an Fv fragment, a disulfide stabilized Fv fragment (dsFv), a (dsFv)₂, a bispecific dsFv (dsFv−dsFv′), a disulfide stabilized diabody (ds diabody), a single-chain antibody molecule (scFv), DARPins, FABs, nanobodies or single chain variable fragments (scFv)).

As described herein, prior to selecting polypeptides or proteins having desired characteristics, the microcapsules produced by encapsulation and solubilization may be contacted with a ligand or substrate that binds to the polypeptide or protein of interest, as conceived in the original CHESS method. However a significant advantage of using eukaryotic cells rather than bacterial cells for encapsulation and use in selection methods described herein is that other means of selecting functional protein mutants can be applied in addition to ligand binding, or as an alternative to ligand binding if no suitable ligand exists. This is because the proteins of interest are expressed in a cell that harbors all the necessary machinery required for the physiological action of the protein.

Accordingly, the methods of the present disclosure include an optional step or steps of contacting the cells and/or said microcapsules with one or more agents to facilitate detection of activity or function of polypeptides or proteins of interest. The one or more agents may comprise ligands, substrates or other biosensors capable of facilitating detection of polypeptide or protein activity or function, such as those hereinbelow described. The contacting step or steps may occur prior to, concurrently with, or subsequent to either or both of the encapsulating and solubilizing steps. By way of example only, the eukaryotic cells employed in the method may harbour a reporter gene expressing a fluorescent protein, the cells may be stimulated with a suitable agonist before encapsulation, such that in the presence of a functional polypeptide or protein of interest, it will switch on the reporter gene and thus the cells would express the fluorescent protein. Where multiple contacting steps are employed, each step may include contacting the cells or microcapsules with the same or different agents, and each step may occur at the same or different times with respect to the encapsulating and solubilizing steps.

As will be appreciated by those skilled in the art, the present disclosure contemplates and encompasses embodiments in which a contacting step is not required in order to detect activity or function of polypeptides or proteins of interest and thereby facilitate selection of polypeptides and proteins of interest having one ore more desired properties. For example, the cells may naturally express, produce or contain (or may have been modified or manipulated prior to employing the method of the present disclosure to express, produce or contain) the ligands, substrates, or other sensors required to facilitate detection of activity or function of polypeptides or proteins of interest. By way of example only, the eukaryotic cell may be engineered to express a fusion protein between a fluorescent protein and a protein or polypeptide capable of interacting with a functional or active polypeptide or protein of interest. In this way, activity or function of the polypeptide or protein of interest may be detected or monitored without the needs for the addition of an exogenous agent. As noted above, this highlights one of the key advantages offered by the present invention in making possible the employment of CHESS and related selection and screening methods in eukaryotic cells.

Ligands that bind a polypeptide or protein of interest and that are suitable for use in CHESS and related methods can be identified by those of skill in the art. In some examples, the ligand contains a detectable label, such as a fluorescent dye (e.g. 4′,6-diamidino-2-phenylindole, dihydrochloride (DAPI), xanthene dyes such as 5- or 6-Carboxyfluorescein (5-FAM and 6-FAM) or Fluorescein, rhodamine dyes such as 5- or 6-Carboxytetramethylrhodamine (5 or 6-TAMRA), and cyanine dyes). In other examples, the ligand is an enzyme substrate, where binding of the protein variant (which is an enzyme in this embodiment) results in the generation of a detectable signal. For example, 3-(4,5-dimethylthiazol-2-yl)-2, 5-diphenyltetrazolium bromide (MTT) can be used as the ligand for variants of fumarate reductase, where the active variants reduce the MTT to insoluble, purple-coloured formazan. Ligands may be nucleic acid molecules, peptides or proteins including, for example, natural ligands of the protein, as well as engineered protein ligands such as antibodies and fragments thereof (e.g. Fab fragment, scFv, sdAb (i.e. nanobodies)).

Alternatively or in addition, selection of functional protein variants and mutants may be based on fluorescence readouts of protein function such as dimerization of the protein, interaction with other cellular proteins, stimulation of cell-signaling pathways, activation of gene transcription, kinase activation, activation of ion channels, activation of protein degradation, internalization of proteins, membrane reorganization, activation of cellular enzymes, and activation of protein trafficking. Such fluorescence readouts may be obtained by, for example, staining cells with fluorescent dyes or reporter dyes, recombinant expression of fluorescent protein-fused proteins, recombinant expression of bimolecular-fluorescent complementation partner fused proteins, recombinant expression of fluorescent protein-fused proteins where the fluorescent proteins are pairs for fluorescent-resonance energy transfer (FRET) detection of protein-protein interactions, recombinant expression of signaling sensors (e.g. CAMYEL FRET sensor for cAMP, GCaMP for calcium, voltage sensors), or reporter genes expressing fluorescent proteins or enzymes.

By way of example only, for the identification and selection of GPCR variants or mutants, FRET donor/acceptor fluorescent protein-GPCR fusion protein may be co-expressed with FRET donor/acceptor fluorescent protein-G proteins or arrestin protein and interactions between the GPCR and these effector proteins monitored using FRET or monitoring receptor dimerization (see, for example, Pfleger and Eidne (2005) Monitoring the formation of dynamic G-protein-coupled receptor-protein complexes in living cells. Biochem J 385:625-637). A range of alternative biosensor-based labelling approaches are known to those skilled in the art, including for example, FRET-based sensors, bioluminescence resonance energy transfer (BRET)-based sensors and lanthanide-based homogeneous time resolved fluorescence (HTRF) sensors. Non-limiting examples of suitable sensors are described, for example, in Tainaka et al (2010) Design strategies of fluorescent biosensors based on macromolecule receptors. Sensors 10:1355-1376.

For example, cells may be labelled with a calcium-sensing dye and receptor-induced calcium signaling monitored, receptor activation of specific genes may be monitored using reporter assays (see, for example, Hill et al. (2001) Reporter-gene systems for the study of G-protein-coupled receptors. Curr Opin Pharmacol 1:526-532), or a FRET based signaling sensor such as CAMYEL may be co-expressed (see, for example, Matthiesen and Nielsen (2011) Cyclic AMP control measured in two compartments in HEK293 cells: phosphodiesterase K(M) is more important than phosphodiesterase localization. PLoS One 6).

Gene libraries encoding variants of a protein can be prepared and transfected or transduced into cells using any method known to those skilled in the art. For example, suitable vectors for use in transducing eukaryotic cells in accordance with the present disclosure include retrovirus vectors, adenovirus vectors and adeno-associated virus vectors. One example of a suitable retrovirus-based system comprises lentiviral vectors and transduction. A number of lentiviral vector and transduction systems suitable for use in accordance with the present disclosure are commercially available and are well known to those skilled in the art. Where small-molecular weight proteins are the protein of interest, they can be produced as fusions to other oligopeptides or proteins to form larger structures (e.g. a triple GFP tag). Thus, gene libraries can in some embodiments include fusion genes that encode fusion proteins.

Methods for producing a diverse library of a gene (i.e. gene diversification) are well known in the art and described elsewhere (see, e.g. Packer and Liu (2015) Methods for the directed evolution of proteins. Nat Rev Genet 16:379-393). Gene diversification can involve random mutagenesis, focused mutagenesis or a combination thereof. These methods include, but are not limited to, chemical or environmental mutagenesis (e.g. nitrous acid, UV irradiation and bisulfite), the use of mutator strains (e.g. XL1-red E. coli), error prone PCR, site directed saturation mutagenesis, homologous recombination (e.g. DNA shuffling, family shuffling, staggered extension process (StEP), random chimeragenesis on transient templates (RACHITT), nucleotide exchange and excision technology (NExT), heritable recombination, assembly of designed oligonucleotides (ADO) and synthetic shuffling) and non-homologous recombination (e.g. incremental truncation for the creation of hybrid enzymes (ITCHY), sequence homology-independent protein recombination (SHIPREC), non-homologous random recombination (NRR), sequence-independent site-directed chimeragenesis (SISDC) and overlap extension PC) (see, reviewed in Packer and Liu (2015) Methods for the directed evolution of proteins. Nat Rev Genet 16:379-393).

The solubilization step disrupts the cell wall or outer membrane and exposes the cell's interior, whereas the coating applied to the cell during the encapsulation step retains structures and molecules in the cell to be probed in subsequent steps. The solubilization step may employ any method that does not disrupt the layers coated onto the cell during the encapsulation step. Non-limiting examples include treatment with a detergent, perforin, lysozyme, mild ultrasonic treatment, hyper-osmotic or hypo-osmotic shock, electroporation, treatment with alcohol or other organic solvent, freeze-thaw cycles, heating and boiling the capsules and pressure gradients. In some particular embodiments, solubilizing the membrane of the encapsulated cells includes exposing the encapsulated cells to a detergent in aqueous solution.

In accordance with embodiments of the present disclosure, sequences encoding selected polypeptides or proteins can be extracted and isolated from capsules using methods well known to those skilled in the art. Such isolated sequences may be subjected to further analysis, including for example subcloning and re-transfection, transduction or transformation into cells to facilitate one or more further rounds of selection or screening, employing methods the subject of the present disclosure or any other suitable method known to those skilled in the art. Prior to such further rounds of selection or screening, the sequences may be mutagenized or otherwise modified or manipulated.

It will be appreciated by persons skilled in the art that numerous variations and/or modifications may be made to the present disclosure without departing from the spirit or scope of the disclosure as broadly described. The present embodiments are, therefore, to be considered in all respects as illustrative and not restrictive.

The present disclosure will now be further described in greater detail by reference to the following specific examples, which should not be construed as in any way limiting the scope of the disclosure.

### EXAMPLES

### Example 1—Encapsulation of Mammalian Cells

PEG diacrylate precursor solution was prepared, containing 25% PEG diacrylate 6K (Sigma 701963) in complete Phenol-red-free DMEM media with 225 mM triethanolamine (TEA, Sigma 90279) and 37 mM 1-vinyl-2-pyrrolidinone (VP, Sigma V3409) at pH 8. The solution was filtered sterilized using 0.22 μm syringe filter and oxygen removed by bubbling with argon for 15 minutes.

Human Embryonic Kidney (HEK) 293T cells expressing GFP and human embryonic kidney cells stably expressing stabilised neurotensin receptor 1 (NTS1) were pelleted at 1500 G for 2 min, the excess medium removed and the pellets stained with 100 μM eosin Y (EY, Sigma E4009) in Phenol-red-free DMEM media for 5 mins. The stained pellets were washed 3 times with Phenol-red-free DMEM media and resuspended in 2 ml PEG diacrylate precursor solution. Cells were aliquoted into 24 well plates and illuminated using a POLARstar Omega plate reader (BMG LabTech), in spectrophotomer mode (broad emission wavelength), for 58 seconds with plate shaking. Encapsulated cells were washed 3 times with Phenol-red-free DMEM media or phosphate buffered saline (PBS).

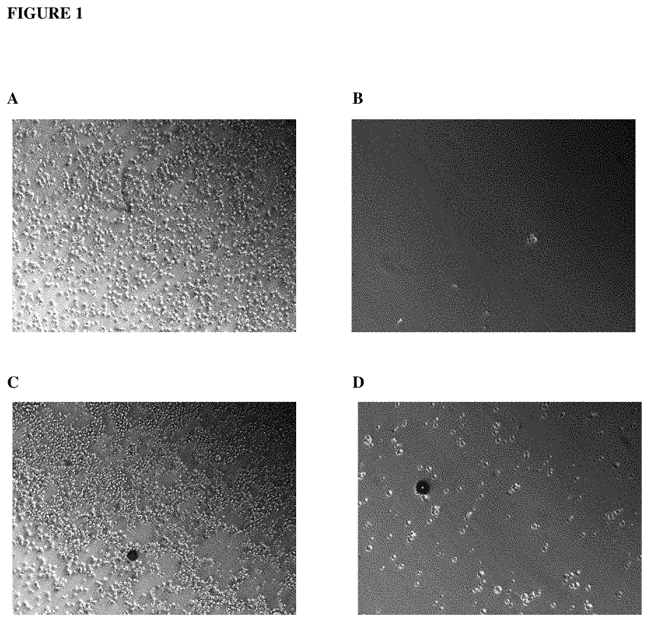

As shown in FIG. 1, light microscopic analysis of HEK cells in the presence of 1% 3-[(3-cholamidopropyl)dimethylammonio]-1-propanesulfonate (CHAPS) demonstrates that the cellular structure and integrity of the encapsulated cells is retained after 24 hours (FIG. 1D), whereas the unencapsulated cells were dissolved (FIG. 1B). Confocal microscopic analysis of encapsulated and unencapsulated HEK cells expressing GFP, in the presence or absence of 1% CHAPS, revealed the same results (data not shown).

### Example 2—Flow Cytometry Analysis of Encapsulated Cells

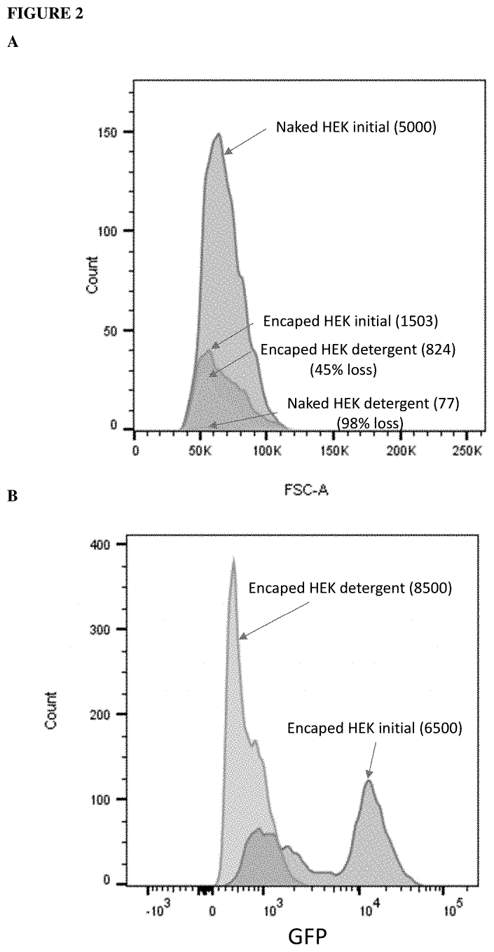

HEK 293T cells stably expressing eGFP were encapsulated as described in Example 1. Samples of non-encapsulated and encapsulated cells were incubated in PBS or PBS with 1% CHAPS at 22° C. for 24 h. Samples were analysed with flow cytometry initially after encapsulation, or after 24 h treatment. The GFP fluorescence of at least 1000 single encapsulated cells was monitored (488 nm excitation, 530 nm±30 nm emission) to monitor the amount of GFP retained within each capsule. Flow cytometry analysis shows that encapsulation resulted in a significantly reduced loss of cells in the presence of detergent than unencapsulated cells (FIG. 2A). The detergent treatment produced “pores” in the cells enabling GFP to leak out (FIG. 2B).

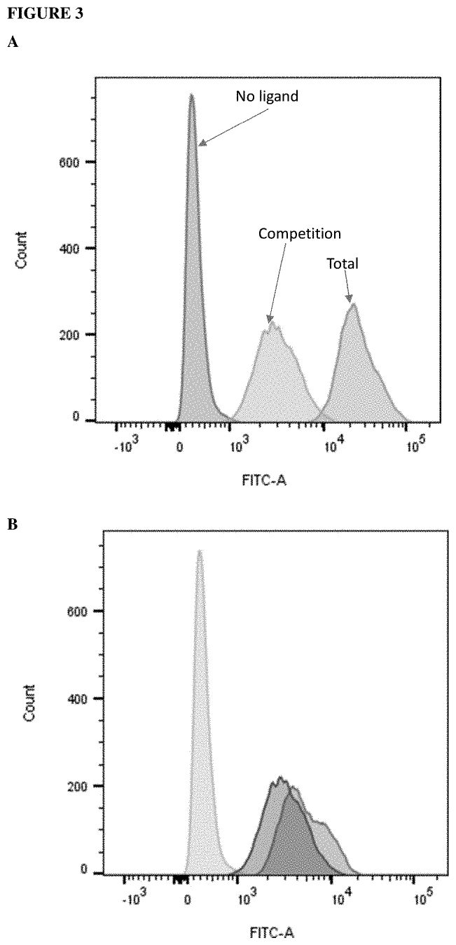

For further receptor binding studies, HEK 293T cells stably expressing a detergent stable neurotensin receptor 1 (a GPCR) (see Scott and Plückthun (2013) Direct molecular evolution of detergent-stable G protein-coupled receptors using polymer encapsulated cells. J. Mol. Biol. 425:662-677) were encapsulated as in Example 1 and incubated in PBS or PBS with 2% n-decyl-β-D-maltopyranoside (DM) for 24 hours at 25° C. Samples were also treated with 100 nM fluorescein labelled neurotensin peptide (FAM-NT8-13), or with 100 nM FAM-NT and 10 μM unlabeled neurotensin peptide (NT8-13) as a competitor. Specific peptide binding to encapsulated cells was determined with flow cytometry, monitoring fluorescein fluorescence (488 nm excitation, 530 nm±30 nm emission), of at least 1000 encapsulated single cells. This analysis demonstrated that neurotensin binding ability is retained in the presence of detergent (FIG. 3B), although the signal generated was reduced due to receptor denaturation by the detergent.

The experiments described herein in Examples 1 and 2 demonstrate that encapsulation of mammalian cells by photopolymerization produces detergent-stable microcapsules that are stable for at least 2 days at 25° C., in which ligand binding to a G protein-coupled receptor can be successfully detected.

### Example 3—Nanobead Co-Encapsulation of Mammalian Cells

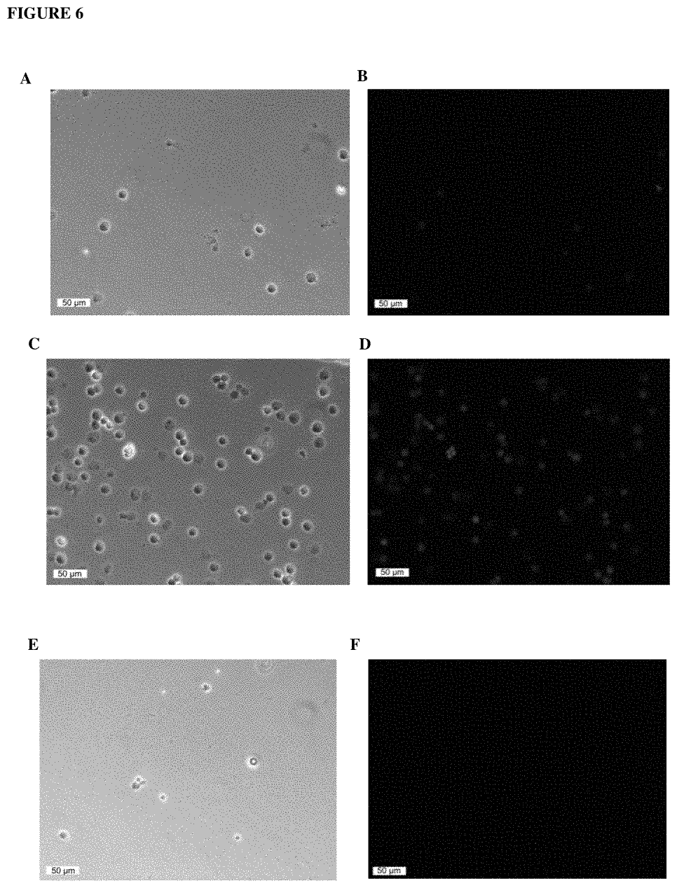

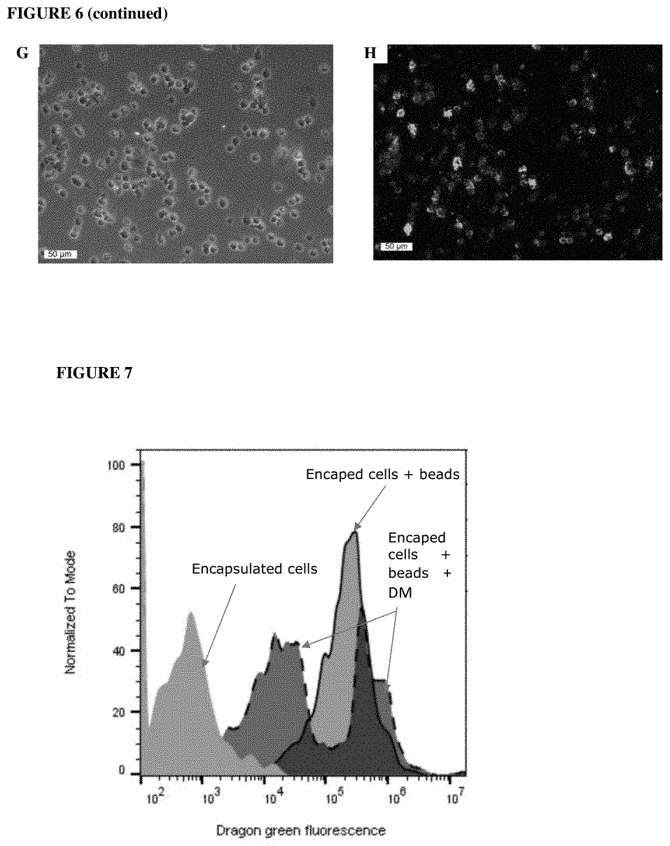

10 μg/mL of dragon green-labelled 200 nm diameter polystyrene beads (nanobeads) were added to the PEG diacrylate-HEK 293-T cell encapsulation mixture obtained as described in Example 1, prior to illumination. The dragon green nanobeads were obtained from Bangs Laboratories Inc.; dragon green is a dye with an excitation wavelength maximum of 501 nm, and an emission wavelength maximum of 510 nm. The mixture was then illuminated and the encapsulated cells washed as described in Example 1. Dragon green fluorescence of at least 5000 single naked cells, of naked cells incubated with dragon-green nanobeads but not encapsulated, of encapsulated cells without nanobeads, and of nanobead co-encapsulated cells was assessed using flow cytometry. The samples analysed by flow cytometry were then fixed with methanol treatment for 5 minutes and mounted on cover slips for analysis by transmitted light and fluorescence microscopy; all microscopy images were acquired with identical settings using a 20× objective. Scale bars in the images of FIGS. 5 and 6 indicate a length of approximately 50 μm.

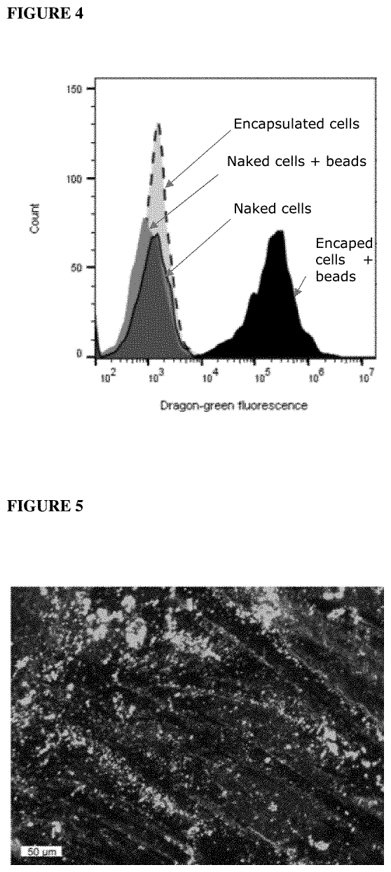

As shown in FIG. 4, dragon-green fluorescence associated with cells which had been co-encapsulated with dragon green nanobeads was observed with flow cytometry (“Encaped cells+beads”). Dragon-green nanobeads alone were not observable with flow cytometry due to their small size (data not shown). In contrast to the co-encapsulated cells, dragon-green fluorescence was not observed for naked cells which had been incubated with 10 μg/mL dragon-green nanobeads without encapsulation (“Naked cells+beads”), nor for naked (unencapsulated) cells (“Naked cells”) or cells encapsulated without dragon-green nanobeads (“Encapsulated cells”).

The fluorescence of dried, aggregated dragon green nanobeads was observed with fluorescence microscopy, as shown in FIG. 5. The nanobeads are too small to be resolved under transmitted light mode. FIG. 6 shows transmitted light and fluorescence microscopy images of the cell samples. Naked cells (FIG. 6A), naked cells incubated with dragon green nanobeads (FIG. 6C), encapsulated cells (FIG. 6E) and cells co-encapsulated with dragon green nanobeads (FIG. 6G) were of similar size (diameter of approximately 15 μm) and shape under transmitted light mode. Dragon green fluorescence was not observed for naked cells (FIG. 6B) or encapsulated cells (FIG. 6F), but some dim fluorescence, although not punctate nanobead-like fluorescence, was associated with naked cells that had been incubated with dragon green nanobeads (FIG. 6D). Strong, punctate fluorescence was localized around cells that had been co-encapsulated with dragon green nanobeads (FIG. 6H).

These analyses demonstrate that the nanobeads were trapped in the encapsulating PEG layer around the cells, and that the nanobeads remained associated with the encapsulated cells even after extensive washing. It is thus possible to validate successful encapsulation and detect successfully encapsulated cells in mixed samples by co-encapsulating with fluorescent nanobeads.

### Example 4—Detergent Treatment after Nanobead Co-Encapsulation

Cells co-encapsulated with dragon green nanobeads as produced in Example 3 were treated with 1% n-dodecyl-β-D-maltopyranoside (DDM) at room temperature for 3 hours (for analysis by flow cytometry) and for 24 hours (for analysis by microscopy). Dragon green fluorescence of at least 5000 single encapsulated cells, of cells co-encapsulated with dragon green nanobeads, and of co-encapsulated cells treated with detergent for 3 hours was assessed by flow cytometry. Methanol fixation of detergent-treated nanobead co-encapsulated cells resulted in dissolution of the capsules (data not shown), so samples treated with detergent for 24 hours were mounted on coverslips without fixation and analysed by transmitted light and fluorescence microscopy.

As shown in FIG. 7, detergent treatment of nanobead co-encapsulated cells resulted in some reduction in the dragon green fluorescence, likely due to a reduction in the number of nanobeads associated with the encapsulated cells after detergent treatment.

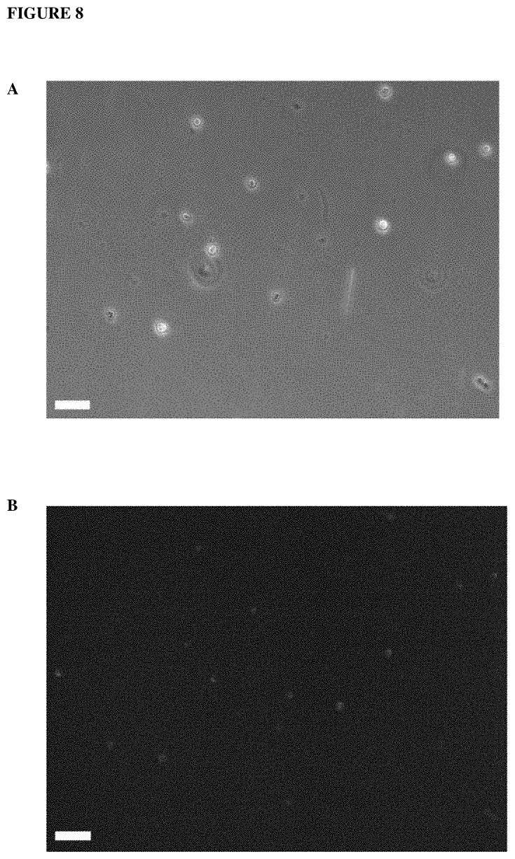

FIG. 8A shows a transmitted light microscopy image of the co-encapsulated cells after 24 hours of treatment with detergent, revealing a population of cell-like capsules. Those cell-like capsules exhibited some dragon green fluorescence as shown in the fluorescence microscopy image of FIG. 8B, although this fluorescence was reduced compared to the co-encapsulated cells which were not treated with detergent (FIG. 6H).

These results indicate that co-encapsulation with nanobeads allows the discrimination of properly formed capsules after detergent treatment and further illustrates that the encapsulation method described here results in detergent-resistant capsules.

### REFERENCES

(1) Baroli, B. Photopolymerization of biomaterials: issues and potentialities in drug delivery, tissue engineering and cell encapsulation technologies, J Chem Technol Biotechnol, 81:491-499 (2006)
(2) Hill, S J. et al. Reporter-gene systems for the study of G-protein-coupled receptors. Curr Opin Pharmacol 1:526-532 (2001)
(3) Matthiesen, K and Nielsen, J. Cyclic AMP control measured in two compartments in HEK293 cells: phosphodiesterase K(M) is more important than phosphodiesterase localization. PLoS One 6 (2011)
(4) Packer, M S and Liu, D R. Methods for the directed evolution of proteins. Nat Rev Genet 16:379-393 (2015)
(5) Pfleger, K D and Eidne, K A. Monitoring the formation of dynamic G-protein-coupled receptor-protein complexes in living cells. Biochem J 385:625-637 (2005).
(6) Scott, D J and Plückthun, A. Direct molecular evolution of detergent-stable G protein-coupled receptors using polymer encapsulated cells. J. Mol. Biol. 425:662-677 (2013)
(7) Tainaka, K et al. Design strategies of fluorescent biosensors based on macromolecule receptors. Sensors 10:1355-1376 (2010)
(8) Yong, K J and Scott, D J. Rapid directed evolution of stabilized proteins with cellular high-throughput encapsulation solubilization and screening (CHESS), Biotechnol Bioeng 112:438-446 (2015)

What is claimed is:

1. A method for selecting polypeptides or proteins having one or more desired properties from a library of sequences expressed in eukaryotic cells, comprising:
(i) encapsulating said cells by photopolymerization;
(ii) solubilizing said encapsulated cells to produce semipermeable microcapsules;
(iii) optionally contacting said cells and/or said microcapsules with one or more agents to facilitate detection of activity or function of polypeptides or proteins of interest; and
(iv) selecting polypeptides or proteins of interest having one or more desired properties.

2. The method according to claim 1, wherein step (iii) occurs prior to step (i) or step (ii), concurrently with step (i) or step (ii), or subsequent to step (ii).

3. The method according to claim 1, wherein said contacting comprises two or more contacting steps, with the same or different agents, and wherein at least one contacting step occurs prior to said encapsulating or solubilizing and at least one contacting step occurs subsequent to said encapsulating or solubilizing.

4. The method according to claim 1, wherein the eukaryotic cells are insect cells or mammalian cells.

5. The method according to claim 1, wherein the photopolymerization comprises photopolymerization of a poly(ethylene glycol) (PEG)-based monomer.

6. The method according to claim 5, wherein the PEG-based monomer is PEG-diacrylate.

7. The method according to claim 1, wherein the photoinitiator comprises a dye.

8. The method according to claim 7, wherein the dye is an eosin dye.

9. The method according to claim 8, wherein the eosin dye is eosin Y.

10. The method according to claim 1, wherein the photopolymerization is carried out in the presence of an amine and/or an accelerator.

11. The method according to claim 10, wherein the amine is triethanolamine.

12. The method according to claim 10, wherein the accelerator is 1-vinyl-2-pyrrolidinone.

13. The method according to claim 1, wherein the photoinitiator system for photopolymerization comprises an eosin dye, triethanolamine and 1-vinyl-2-pyrrolidinone.

14. The method according to claim 1, wherein the encapsulation step comprises:
treating the cells with the photoinitiator, optionally eosin Y;
washing the cells;
treating the cells with a solution comprising a photopolymerizable residue, optionally a PEG-diacrylate, in the presence of an amine, optionally triethanolamine; and
exposing the cells to a light source, optionally in the presence of an accelerator such as 1-vinyl-2-pyrrolidinone, to encapsulate the cells.

15. The method according to claim 1, further comprising the step of subjecting the microcapsules to one or more environmental conditions prior to the selecting step.

16. The method according to claim 15, wherein the one or more environmental conditions are selected from detergent treatment, temperature, chemical denaturant, or pH.

17. The method according to claim 1, wherein the one or more desired properties comprise stability under one or more environmental conditions.

18. The method according to claim 17, wherein the stability comprises detergent stability, thermostability, chemical stability, or pH stability.

19. The method according to claim 1, wherein the solubilization step comprises treating the encapsulated cells with one or more detergents.

20. A method for encapsulating one or more eukaryotic cells for use in a method for selecting polypeptides or proteins having one or more desired properties from a library of sequences expressed in said eukaryotic cells, wherein said encapsulating comprises photopolymerization.
